SEQUENCE LISTING (11 sequences)

SEQ ID NO: 1 - protein, 5 aa, Artificial Sequence
  CDRH1
DNGMA

SEQ ID NO: 2 - protein, 17 aa, Artificial Sequence
  CDRH2
FISNLAYSIDYADTVTG

SEQ ID NO: 3 - protein, 6 aa, Artificial Sequence
  CDRH3
GTWFAY

SEQ ID NO: 4 - protein, 16 aa, Artificial Sequence
  CDRL1
RVSQSLLHSNGYTYLH

SEQ ID NO: 5 - protein, 7 aa, Artificial Sequence
  CDRL2
KVSNRFS

SEQ ID NO: 6 - protein, 9 aa, Artificial Sequence
  CDRL3
SQTRHVPYT

SEQ ID NO: 7 - protein, 112 aa, Artificial Sequence
  CAA51135 light chain acceptor framework V region amino acid sequence.
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPWTFGQGTKVEIK

SEQ ID NO: 8 - protein, 115 aa, Artificial Sequence
  Humanised heavy chain V region variant H2 , amino acid sequence.
EVQLVESGGGLVQPGGSLRLSCAVSGFTFSDNGMAWVRQAPGKGLEWVSFISNLAYSIDYADTVTGRFTISRDNAKNSLYLQMNSLRAEDTAVYYCVSGTWFAYWGQGTLVTVSS

SEQ ID NO: 9 - protein, 112 aa, Artificial Sequence
  Humanised light chain V region variant L1 amino acid sequence.
DIVMTQSPLSLPVTPGEPASISCRVSQSLLHSNGYTYLHWYLQKPGQSPQLLIYKVSNRFSGVPDRFSGSGSGTDFTLKISRVEAEDVGVYYCSQTRHVPYTFGGGTKVEIK

SEQ ID NO: 10 - protein, 443 aa, Artificial Sequence
  Mature H2 heavy chain amino acid sequence.
EVQLVESGGGLVQPGGSLRLSCAVSGFTFSDNGMAWVRQAPGKGLEWVSFISNLAYSIDYADTVTGRFTISRDNAKNSLYLQMNSLRAEDTAVYYCVSGTWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVWDVSHEDPEVKFNWYVDGVEVHNAKTKPREEQYNSTYRWSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 219 aa, Artificial Sequence
  Mature Light chain amino acid sequence.
DIVMTQSPLSLPVTPGEPASISCRVSQSLLHSNGYTYLHWYLQKPGQSPQLLIYKVSNRFSGVPDRFSGSGSGTDFTLKISRVEAEDVGVYYCSQTRHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2017/0285024 A1
(43) Pub. Date: Oct. 5, 2017

(54) COMPLIMENT FACTOR I BIOASSAY

(71) Applicant: GLAXOSMITHKLINE INTELLECTUAL PROPERTY (NO.2) LIMITED, Brentford (GB)

(72) Inventor: Francisco LOPEZ, King of Prussia, PA (US)

(21) Appl. No.: 15/587,533

(22) Filed: May 5, 2017

Related U.S. Application Data
(63) Continuation of application No. 14/899,144, filed on Dec. 17, 2015.
(60) Provisional application No. 61/836,764, filed on Jun. 19, 2013.

(51) Int. Cl. G01N 33/564; C07K 16/18

(52) U.S. Cl. CPC G01N 33/564; C07K 2317/92; G01N 2800/7042; G01N 2333/988; C07K 16/18; G01N 2500/02; G01N 2800/2821; G01N 2333/4716; G01N 2500/20; C07K 2317/20; G01N 2333/96433

(57) ABSTRACT

The present invention relates to a CFI bioactivity assay designed to quantitatively measure (a) CFI bioactivity of plasma or other body fluids; and (b) bioactivity of CFI in plasma or other body fluids of a human patient afflicted by a disease that involves amyloid deposition in tissues, in particular Alzheimer disease, AMD, glaucoma, or beta-amyloid cataract formation.

### FIELD OF INVENTION

The present invention relates to a CFI bioactivity assay designed to quantitatively measure (a) CFI bioactivity of plasma or other body fluids; and (b) bioactivity of CFI in plasma or other body fluids of a human patient afflicted by a disease that involves amyloid deposition in tissues, in particular Alzheimer disease, AMD, glaucoma, or beta-amyloid cataract formation.

### BACKGROUND OF INVENTION

Complement activation, in particular, the alternative pathway of the complement cascade is believed to be central to the pathogenesis of Age-related Macular Degeneration (AMD). Complement factor I (CFI) and factor H (CFH) play important roles in controlling alternative pathway activation and amplification.

Factor H is an abundant 150-kDa glycoprotein with an average concentration of 500 μg/mL in circulation (Rodriguez de Cordoba et al., 2004). Factor I is an 88-kDa heterodimeric serine protease with a serum concentration of approximately 39-100 μg/ml (De Paula et al., 2003). Complement factor H is the main inhibitor of the alternative pathway in the fluid phase and on surfaces by various mechanisms. The role of factor I is to regulate the activities of the C3 and C5 convertase by proteolytic cleavage of the C3b and C4b in the presence of appropriate cofactors. One important function of factor H is to act as an essential cofactor for factor I in the fluid phase to inactivate C3b, leading to the formation of the inactivated C3b, iC3b. Hence, through their actions on C3b, both factors inhibit C3 convertase formation in the alternative pathway. An in vitro cofactor assay using Western blot has been used to measure the activity of either factor H or factor I in fluid phase in the presence of C3b (Brandstätter et al., 2012).

One of the earliest clinical hallmarks and risk factors of AMD is the formation of subretinal extracellular protein deposits, known as drusen. Among many other proteins in drusen, beta-amyloid protein is believed to be one of the primary stimuli that cause the development of AMD. However, the mechanism of the development of AMD from drusen has not been precisely determined.

Complement activation by beta-amyloid peptides (BAM or Aβ) has been proposed to be one of the important mechanisms underlying the disease progression. Bradt et al. (1998) observed that the addition of fibrillar BAM1-40 and BAM1-42 to a complement source, such as serum or complement protein mixtures, led to the generation of covalent ester-linked complexes of BAM with C3 activation fragments, providing the 1^{st} direct evidence for complement activation by BAM. More recently, the involvement of BAM in complement activation was further demonstrated in a study by Wang et al (2008), where BAM1-40 was shown to bind to CFI and CFH which inhibited the ability of CFI to cleave C3b to iC3b in the cofactor assay, which was revealed by the lack of visible iC3b signal in a Western blot.

### SUMMARY OF INVENTION

In one embodiment, this invention relates to a method of quantitatively measuring the CFI bioactivity of a human body fluid or the bioactivity of CFI in a human body fluid comprising quantitatively measuring the ability of body fluid or CFI to convert C3b to iC3b. Furthermore, this invention also allows one to quantitatively measure the CFI bioactivity of either purified or recombinant CFI comprising quantitatively measuring the ability of purified or recombinant CFI to convert C3b to iC3b.

In a second embodiment, the invention relates to a method of quantitatively measuring the CFI bioactivity of a human body fluid or the bioactivity of CFI in a human body fluid comprising the steps of

(a) diluting the human body fluid with a diluent (for example PBS);
(b) adding CFH and C3b to the diluted human fluid;
(c) incubating the resultant solution of step (b); and
(d) measuring the amount of iC3b generated.

In a third embodiment, the human body fluid is plasma.

In a fourth embodiment

(a) the plasma is diluted about 200-250 fold in PBS and then further diluted in 2× series in PBS;
(b) 6 μL of CFH and C3b is added to the plasma to reach final concentration of 80 μg/mL each; and
(c) incubation of step (c) in second embodiment is carried out at 37° C. for 2 hr, and
(d) amount of iC3b is measured using ELISA.

In a fifth embodiment, the CFI bioactivity of human plasma or body fluid is measured in terms of EC50 which is volume of plasma or body fluid needed to convert 50% C3b into iC3b.

In a six embodiment, the CFI bioactivity of human plasma or body fluid is measured in mU per volume, in which mU is defined as the inverse of the volume of plasma or body fluid in nL (EC50) needed to convert 50%/C3b into iC3b.

In a seventh embodiment, the bioactivity of CFI in plasma or body fluid is derived in terms of Units per amount of μg of CFI using the following equation of 1000/(EC50 (in nL)×[CFI μg/ml]).

In an eighth embodiment, the invention relates to a method of measuring the batch to batch potency of an anti-BAM antibody wherein said method comprises the steps of:
(a) incubating a first batch of an anti-BAM antibody with BAM;
(b) incubating the BAM from step (a) with CFI;
(c) adding CFH and C3b to the mixture of step (b);
(d) measuring the amount of iC3b generated
(e) using the amount of iC3b generated to determine the CFI bioactivity of the first batch;
(f) repeating steps a) to e) for a second batch of an anti-BAM antibody and
(g) comparing the CFI bioactivity of a first batch of anti-BAM antibody to a second batch of anti-SAM antibody in order to determine the potency of anti-BAM antibodies batch to batch, wherein the amount of reagents used the first batch determination is the same as the amount used in the second batch determination, respectively.

In a ninth embodiment, the present invention relates to a method of treating a disease that involves amyloid deposition in tissues of a human patient, comprising the steps of:
a) measuring the CFI bioactivity of a body fluid or the bioactivity of CFI in a body fluid of the patient; and
b) subsequent to measuring the CFI bioactivity, providing to a patient who has lower than a pre-defined amount of CFI bioactivity, an amount of anti-BAM antibody effective to treat the disease.

In a tenth embodiment, the disease is Alzheimer's disease, AMD, glaucoma, or beta-amyloid cataract formation.

In an eleventh embodiment, the present invention relates to an anti-BAM antibody for use in treating a disease that involves amyloid deposition in tissues in a human patient, comprising the steps of:
a) measuring the CFI bioactivity of a body fluid or the bioactivity of CFI in a body fluid of the patient; and
b) subsequent to measuring the CFI bioactivity, providing to a patient who has lower than a pre-defined amount of CFI bioactivity, an amount of anti-BAM antibody effective to treat the disease.

In a twelfth embodiment, the disease is Alzheimer's disease, AMD, glaucoma, or beta-amyloid cataract formation.

In a twelfth embodiment the invention relates to a method of quantitatively measuring the CFI bioactivity of either purified or recombinant CFI comprising the steps of:

(a) diluting the purified or recombinant CFI with a diluent;
(b) adding CFH and C3b to the CFI solution made in step (a);
(c) incubating the resultant solution of step (b); and
(d) measuring the amount of iC3b generated.

### DESCRIPTION OF FIGURES

FIG. 1 Boiling Promoted Inhibitory Activity of BAM1-40. BAM1-40 solution aged for 8 days was boiled for 15 min and tested in cofactor assays.

FIG. 2 Strong CFI-Inhibitory Activity of BAM1-42 Aged for 8 Days, not the Similarly Aged BAM1-40 or Freshly Made BAM1-42. Both of BAM 1-40 and BAM1-42 (Lot 1) solutions aged for 8 days were tested along with freshly made BAM1-42 (Lot 2) solution in cofactor assays.

FIG. 3 Sonication Promoted the Inhibitory Activity of BAM1-42. BAM1-42 solution (Lot 2) was sonicated on day 9, aged further for 8 days, and was tested in a cofactor assay, where 10 μg/mL CFI, 10 μg/mL CFH, and 80 μg/mL C3b were used. BAM1-42 (Lot 2) without sonication and BAM 1-40 at day 25 were also tested.

FIG. 4 Inhibition of CFI Bioactivity Curves by BAM1-42.

FIG. 5 CFI Bioactivity in Human Plasma (1st Trial for Sample HPL8). Human plasma samples were diluted in 200 fold in PBS and then further diluted in 2× series in PBS. Three microliters of the diluted plasma were then mixed with 6 μL of CFH and C3b mixture at a final concentration of 80 μg/mL for each. The mixture was incubated at 37° C. for 2 hr, and the iC3b in the reactions was evaluated in iC3b ELISA kit. As controls, the same amount of plasma was also tested for the amount of iC3b without exogenous C3b and CFH. The volume (nL) of plasma used in each reaction vs iC3b curve was analyzed using a reparametrized 4-parameter logistic equation (Ghosh et al, 1998). The CFI bioactivity in the plasma sample was shown as both the volume and the amount of CFI protein.

FIG. 6 CFI Bioactivity in Human Plasma (2nd Trial for Sample HPL8). Human plasma sample, HPL8, was diluted in 250 fold in PBS and further diluted in 2× series in PBS. Five microliters of the diluted plasma were then mixed with 5 μL of CFH and C3b mixture at a final concentration of 80 μg/mL for each. The mixture was incubated at 37° C. for 2 hr and then the iC3b in the reactions was evaluated in iC3b ELISA kit. The volume (nL) of plasma used in each reaction vs iC3b curve was analyzed using a reparametrized 4-parameter logistic equation (Ghosh et al, 1998). The CFI bioactivity in the plasma sample was shown as both the volume and the amount of CFI protein (3.2 nl and 3.4 U/μg CFI).

FIG. 7 CFI Bioactivity in Mouse Plasma in Cofactor Assay Using Human CFH and C3b. The square symbol denotes the bioactivity of 10 nL of the plasma that was heated to 60° C. for 2 hr prior to assay. This demonstrated that CFI bioactivity is temperature sensitive.

FIG. 8 A typical BAM 1-42 Dose Response in Inhibition of CFI in Cofactor Assay. BAM 1-42, lot 3, was pre-incubated with 1000 ng/mL CFI at 37° C. for 1 hr. Then C3b at 80 μg/mL and CFH at 80 μg/mL were added to the mixture and incubated for another 30 min. The amount of iC3b produced was quantified in the ELISA method. The BAM vs iC3b curve was fitted with Eadie-Hofstee inhibition model using Excel XLfit program. The IC50 for BAM in this assay was 2.06 μM.

FIG. 9 6E10 and 4G8 Blockade of BAM Inhibition of CFI (1st Set). Two clones of anti-BAM antibodies, 6E10 (mIgG1 isotype), and 4G8 (mIgG2b isotype), were tested for inhibition of CFI bioactivity in cofactor assay. BAM at 20 μM was pre-incubated with the anti-BAM antibodies, as well as the isotype controls mIgG1 and mIgG2b at 300 μg/mL at RT for 30 min., then CFI at 1 μg/mL was added and mixed. The Ab/BAM/CFI mixture was incubated at 37° C. for 30 min. CFH at 80 μg/ml and C3b at 80 μg/mL were added and the incubation was continued for 60 min. The amount of iC3b produced was detected by the ELISA method. The reactions were set up in triplicates. The concentration of antibody vs iC3b curve was analyzed using a reparametrized 4-parameter logistic equation (Ghosh et al, 1998).

FIG. 10 6E10 and 4G8 Blockade of BAM Inhibition of CFI (2nd Set). Anti-BAM antibodies, 6E10 (mIgG1 isotype), and 4G8 (mIgG2b isotype), were tested for inhibition of CFI bioactivity in the cofactor assay. BAM at 20 μM was pre-incubated with the anti-BAM antibodies, as well as 200 μg/mL isotype controls 11A50-B10 and mIgG2b at RT for 5-10 min. Clone of 11A50-B10 is also an anti-BAM antibody, but does not inhibit CFI (See FIG. 2). Therefore it was served as a mouse isotype control for 6E10 in this study. CFI at 1 μg/mL was then added to the BAM/Ab mixture and mixed. The Ab/BAM/CFI mixture was incubated at 37° C. for 30-40 min. CFH at 80 μg/ml and C3b at 80 μg/mL were added and the incubation was continued for 40-50 min. The amount of iC3b produced was detected by the ELISA method. The all reactions were set up in triplicates. The concentration of antibody vs iC3b curve was analyzed using Excel XLfit Eadie-Hofstee Model.

FIG. 11 Effect of Tween 20, Triton X-100 and Guanidine on BAM Activity. The effect of Tween 20, Triton X-100, and Guanidine on the activity of BAM was tested in the cofactor assay, where 1 μg/mL of CFI and 10 μM BAM was incubated at 37° C. for 40 min in the presence of the treating agents, then 80 μg/mL CFH and 80 μg/mL C3b was added and the reactions were lasted for 1 hr 20 min at 37° C. The iC3b concentration in the reaction was then measured using the ELISA method.

FIG. 12 CFI bioactivity curve in the vitreous fluid of an AMD patient is shifted to the right, when compared to CFI bioactivity curves of patients without AMD. Diluted vitreous samples (5 μl) were mixed with 5 μL of a mixture of 160 mg/mL CFH and 160 mg/mL C3b, and incubated at 37° C. for 2 hr. The iC3b concentration was determined in ELISA. In addition, to correct for amount of CFI, sample levels of CFI were determined by an ELISA. The levels were: 364.65, 1,363.75 and 817.36 ng/ml for the AMD (AMD) and the two non-AMD sample controls (CONT1 and CONT2). Numbers between parentheses indicate the 95% confidence limits for the estimates EC50s.

FIG. 13. ELISAs from three runs evaluating purified (CompTech) and recombinant (GSK) CFI.

FIG. 14. Evaluation of bioactivity for purified (CompTech) and recombinant (GSK) CFI.

FIG. 15. CFI bioactivity in samples from Alzheimer's disease patients treated with anti-BAM (Compound A).

### DETAILED DESCRIPTION

It is known that complement factor I (CFI) and beta-amyloid peptides (BAM) are two important factors involved in pathogenesis of Age-related Macular Degeneration (AMD). To further define the relationship between BAM and the bioactivity of CFI, first we established the in vitro cofactor assay for measuring the bioactivity of CFI. This assay uses an ELISA method for quantitative detection of iC3b, instead of Western blots referred to in the Background Section. In this study, we performed a series of tests to investigate whether BAM1-40 and BAM1-42 affect the enzymatic bioactivity of CFI in the cofactor assay and if the relationship between BAM and bioactivity of CFI is quantitatively measurable. Given the fact that BAM and CFI are the two important contributors of the development of AMD, it is conceivable that if there is an assay to precisely quantify their relationship, the potential application of the assay would be significant. For example, the assay could become a biomarker assay for measuring changes of CFI bioactivity during the course of therapeutic intervention.

Our copending patent application WO2009/040336 and WO2007/113172 teach antibodies to treat AMD, glaucoma, beta-amyloid cataract formation, Alzheimer's disease, among others. WO2009/040336 and WO2007/113172 are herein incorporated by reference in their entirety. Of particular interest are antibodies described in WO2009/040336 comprising the following CDRs.

CDRH1:
(SEQ ID No: 1)
DNGMA
CDRH2:
(SEQ ID No: 2)
FISNLAYSI DYADTVTG
CDRH3:
(SEQ ID No: 3)
GTWFAY

within a human heavy chain variable region originating from the VH3 gene family and:

CDRL1:
(SEQ ID NO: 4)
RVSQSLLHSNGYTYLH
CDRL2:
(SEQ ID No: 5)
KVSNRFS
CDRL3:
(SEQ ID No: 6)
SQTRHVPYT

within a human light chain variable region originating from the amino acid sequence disclosed in GenPept entry CAA51 135 (SEQ ID No:7).

Also of particular interest is an antibody having a heavy chain variable region having the sequence set forth in SEQ ID NO:8 and a light chain variable region having the sequence set forth in SEQ ID NO: 9.

Further of particular interest is an antibody comprising a heavy chain having the sequence set forth in SEQ ID NO: 10 and a light chain having the sequence set forth in SEQ ID NO: 11 (herein defined as Compound A)

The assays of the present invention are shown to be useful as biomarker assay for measuring changes of CFI bioactivity during the course of using the above antibodies. Moreover the present assays equally are not limited to antibodies disclosed in WO2009/040336 and WO2007/113172 but can be applied to any antibodies whose mechanism is to treat a disease that involves amyloid deposition in tissues (in particular Alzheimer's disease, AMD, glaucoma, or beta-amyloid cataract formation) through restoring CFI bioactivity suppressed by BAM.

We have now shown that BAM 1-42 reduces the CFI bioactivity as demonstrated by a reduced ability of CFI to convert C3b into iC3b in a cofactor assay. Subsequently, we have (a) demonstrated that anti-BAM antibody can reverse or restore the CFI bioactivity that has been inhibited by BAM; and (b) we have developed a biomarker assay to quantify the changes in CFI bioactivity in human and mouse plasma during or after anti-BAM therapy. In other words, the present CFI quantitative bioactivity assay can be used to ascertain if a human patient afflicted by disease that involves amyloid deposition in tissues (in particular Alzheimer's disease, AMD, glaucoma, or beta-amyloid cataract formation) would benefit from receiving anti-BAM antibodies by first determining the level of CFI bioactivity in his/her body fluids or CFI in body fluids. Also this assay can be used to determine if a patient who received anti-BAM antibody therapy has had a positive effect by determining the level of CFI bioactivity in his/her body fluids or CFI in body fluids by comparing before and after anti-BAM antibody was administered. The present CFI biomarker assays are preferably done in vitro using human body fluid (e.g. plasma) samples as described herein.

Typically, the present CFI bioactivity assay can be accomplished by the method comprising the steps of

(a) diluting the human body fluid with a diluent (for example PBS);
(b) adding CFH and C3b to the diluted human fluid;
(c) incubating the resultant solution of step (b); and
(d) measuring the amount of iC3b generated.

We have also demonstrated that the CFI bioactivity assay can be used to measure batch-to-batch potency of anti-BAM antibodies by the method comprising the steps of:

(a) incubating an anti-BAM antibody with BAM;
(b) incubating the BAM from step (a) with CFI;
(c) adding CFH and C3b to the mixture of step (b);
(d) measuring the amount of iC3b converted to measure the CFI bioactivity; and
(e) comparing the CFI bioactivity batch-to-batch of the anti-BAM antibody in order to correlate the potency of anti-BAM antibodies batch-to-batch.

As used herein “CFI bioactivity” and “bioactivity of CFI” mean the same thing and are obtained as follows:

In one embodiment, CFI bioactivity of human plasma or body fluid is measured in terms of EC50 which is volume of plasma or body fluid needed to convert 50% C3b into iC3b. In one embodiment the volume is measure in nL.

In another embodiment, the CFI bioactivity of human plasma or body fluid is measured as inverse of EC50. For example in one method, it can be measured as mU per volume, in which mU is defined as the inverse of the volume of plasma or body fluid in nL (EC50) needed to convert 50%/o C3b into iC3b.

In another embodiment, the bioactivity of CFI in plasma or body fluid is derived in terms of Units per amount of μg of CFI using the following equation of 1000/(EC50 (in nL)×[CFI μg/ml]).

### Examples

Part I—Feasibility Study for Establishing ELISA Assay for Measuring CFI Bioactivity Source of Materials

Factor I, Factor H, C3b, and iC3b proteins were purchased from Complement Technology Inc (Tyler, Tex.). MicroVue iC3b EIA kit (ELISA kit) was purchased from Quidel Corp (Santa Clara, Calif.). Amyloid Beta-Protein (1-40) (HCl form) and Amyloid Beta-Protein (1-42) (TFA form) were purchased from Peptides International, Inc. (Louisville, Ky.).

### 1.1 Preparation of Standards

The reference standard iC3b (1.1 mg/mL) was used to prepare the standards at the nominal concentrations of 0.01, 0.03, 0.1, 0.3, 1, 3, and 10 μg/mL in Assay Buffer (10 mM Tris, 60 mM NaCl, 0.1% BSA, 0.1% Tween 20, pH 7.2) as assay matrix.

### 1.2. Preparation of BAM Solutions

BAM 1-40 at 1 mM: A whole vial of the amyloid-beta-protein (1-40) in HCl form containing 0.55 mg was dissolved in 127 μL PBS by sonication in Branson 1210 sonicator for 15 min. The solution was stored at 4° C.

BAM 1-42 in TFA form at 1 mM (Lot 1): A whole vial of the amyloid-beta-protein (1-42) containing 0.56 mg was suspended in 124 μL PBS by sonication for 15 min. The BAM1-42 in suspension was attempted for dissolution by using 1/10 volume of DMSO, which was not successful. The suspension was finally dissolved by adjusting pH to ˜7 using NaOH and HCl (for a total of 90 μL BAM, 6 μL 0.5N NaOH, 2 μL 1N HCl was used). The final 1 mM solution was stored at 4° C. Another vial of BAM 1-42 (Lot 2) was made into 1 mM solution by directly dissolving in 112 μL 1 mM NaOH, then 12 μL of 10×PBS was added to make the final solution in 1×PBS. An aliquot of the Lot 2 BAM1-42 solution was sonicated for 15 min in the sonicator after storage at 4° C. for 9 days. Both sonicated and non-sonicated BAM 1-42 solutions (Lot 2) were stored at 4° C.

### 1.3. In Vitro Cofactor Assay

BAM 1-40 or BAM 1-42 (0-400 μM) were mixed with CFI (0-30 μg/mL) and pre-incubated at 37° C. for 30 to 60 min. A mixture of CFH (10-80 μg/mL) and C3b (80-150 μg/mL) was added into the tubes containing the CFI or BAM/CFI mixture. After a 30 minute-incubation at 37° C., the reaction mixtures were directly diluted into Assay Buffer and used for ELISA detection of iC3b.

1.4. ELISA Detection of iC3b

The detailed procedures other than indicated below were performed following the instruction provided with the ELISA kit. The diluted reaction mixtures and standards were applied to the 8-well strips at 60 μL per well and incubated at RT for 30 min. After washing 5 times in a plate washer with an in house-made washing buffer (10 mM Tris, 60 mM NaCl, 0.1% Tween 20, pH7.2), the 8-well strips were loaded with 50 μL of the HRP-anti-human iC3b conjugates per well and incubated at RT for 30 min. At the end of incubation, the 8-well strips were washed in the plate washer 5 times and incubated with 100 μL of the HRP substrates provided in the kit for additional 30 min. The reaction was stopped by addition of 50 μL Stop Buffer. The absorption at 405 nm was measured within 10 minutes.

### 1.5. Major Computer Systems and Data Processing

Absorption data were acquired using a BioTek ELx800 microplate reader (Winooski, Vt.), which was controlled by a Dell PC workstation via Gen5™ software (BioTek). The acquired data were processed using Gen5 software (BioTek) and Microsoft Excel 2003. A standard curve for iC3b quantification was constructed by plotting the concentrations of standards in log scale (X-axis) versus their corresponding absorption at 405 nm (Y-axis, OD405), and fitted with a 4-parameter logistic algorithm. The concentration of the iC3b in the samples was determined based on the standard curve. Microsoft XLfit was used to graph the curves of the concentrations of iC3b versus the concentrations of BAM or CFI.

### 2. Results and Discussion of the Feasibility Study

2.2 Measurement of iC3b, the End Product of Cofactor Assays

The ELISA method was used to quantify the concentration of the end product, iC3b, of the CFI-cleaved C3b in a cofactor assay, which was also the measurement for the bioactivity of CFI. The direct readout of the ELISA detection was OD405, which was then converted into the concentrations of iC3b based on iC3b standards used in the same ELISA plate. For qualitative measurement of the CFI bioactivity, OD405 was sometimes used directly without conversion into the iC3b concentration even though the relationship between OD405 and the iC3b concentrations was non-linear, but correlated. When iC3b standards were used, a typical iC3b standard curve with a range of 0.03-3 μg/mL was obtained.

### 2.2 Titration of CFI in Cofactor Assays

In order to test the effect of BAM on the bioactivity of CFI, an appropriate range of concentrations of CFI was needed. CFI at concentrations spanning 1 μg/mL to 10 μg/mL were first tested at an incremental of 1 logarithm for a total of 8-logarithms. Based on the 8-log test results, CFI at concentrations ranging from 0.3 ng/mL to 1000 ng/mL were tested further at an incremental of half-logarithm in the cofactor assay. An optimal concentration range of CFI between 3 to 10,000 ng/mL was selected for testing the effect of BAM in next series of experiments.

### 2.3. Effect of BAM at Day 0 on the Bioactivity of CFI

Based on the methods described in Wang et al. (2008), BAM 1-40 in HCl form was used and dissolved by sonication in PBS. Similarly, sonication in PBS was tried initially to dissolve BAM1-42, but failed. The BAM1-42 finally was dissolved by adjusting pH. The freshly made BAM1-40 solution and BAM1-42 suspension (day 0) were tested in cofactor assays on the bioactivity of CFI in the concentration range suggested from the previous experiment. Since neither BAM shifted the CFI dose response curves, both BAMs at day 0 were concluded to be inactive in inhibiting CFI bioactivity despite the reduced iC3b production in BAM-treated CFI at higher concentrations compared to the PBS control. Such reduction was probably due to technical reasons, such as higher dilution factors and single test points.

### 2.4 Effect of Aged BAM on the Bioactivity of CFI

A vast amount of literature has shown that BAMs aggregate into oligomers with increased activity (Uversky, 2010; Bertoncini and Celej, 2011, Straub and Thirumalai, 2011). Therefore, it was reasoned that BAM solution, whose activity was undetectable at day 0, may increase its activity with aging. Both BAM solutions that were stored at 4° C. from the previous experiments were tested again after aging for 2 and 3 days in cofactor assays. It is known that the concentrations of CFI and CFH in the cofactor assay contributed significantly to the efficiency of cleavage of C3b into iC3b (Brandstätter et al., 2012). In order to detect the potential inhibitory effect of BAM in a wide range, especially in the low activity end, five different cofactor assay conditions with different iC3b conversion efficiencies were used:

1 μg/mL CFI, 10 μg/mL CFH (Low iC3b Conversion Efficiency)

1 μg/mL CFI, 30 μg/mL CFH (Medium Low iC3b Conversion Efficiency)

10 μg/mL CFI, 10 μg/mL CFH (Medium iC3b Conversion Efficiency)

10 μg/mL CFI, 30 μg/mL CFH (Medium High iC3b Conversion Efficiency)

10 μg/mL CFI, 80 μg/mL CFH (High iC3b Conversion Efficiency)

Results from three separate experiments consistently showed that BAM 1-40 and 1-42 solutions aged for either 2 or 3 days possessed significantly higher CFI inhibitory activity than those without aging. It is concluded that (1) BAM 1-42 was much more active at inhibiting CFI bioactivity than BAM1-40 (Table 1) and (2) cofactor assay conditions indeed affected the efficiency of BAM-mediated inhibition of CFI bioactivity. Therefore, the BAM1-42 at day 3 was chosen for identifying its IC50 under low, medium and high iC3b conversion efficiency conditions. The IC50s for BAM 1-42 at low, medium, and high efficiency conditions were 1.5 μM, 31.2 μM, and 60.1 μM, respectively.

### 2.5 is BAM1-42 True Inhibitor of CFI Bioactivity?

To answer whether BAM1-42 is a true inhibitor of CFI bioactivity, a negative control needs to be tested in cofactor assays to demonstrate specificity. To search for the negative controls, two approaches were employed. First, boiling was chosen to treat BAM1-42 and BAM1-40, because boiling usually destroys the biological activity of large molecules. Second, since we have shown that ageing promoted BAM inhibitory activities and the freshly made BAM didn't exhibit detectable activity in previous experiments, a new BAM1-42 solution (Lot 2) was made and used freshly as a negative control along with BAM1-42 (Lot 1) aged for 8 days.

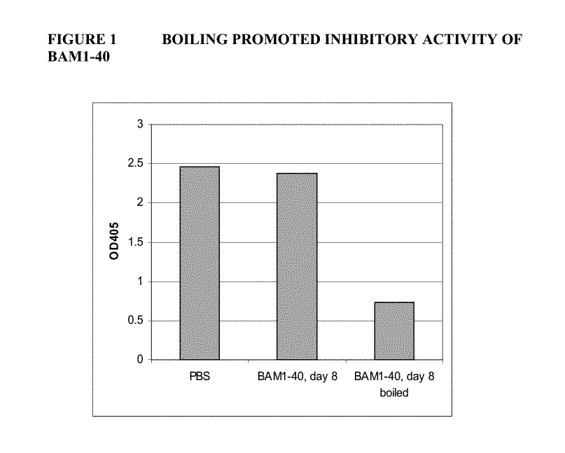

Unexpectedly, boiling for 15 min significantly enhanced the inhibitory activity of BAM1-40 against CFI (FIG. 1), whereas it didn't change significantly the activity of BAM1-42 (not shown). Even though boiling of BAM was not documented as a way to enhance the activity of BAM, it is consistent with fact that higher temperature promotes beta-sheet formation of BAM (Jiang et al., 2012). Therefore, it was logical to hypothesize that boiling might also enhance BAM1-42 activity. Since the activity of BAM 1-42 had reached almost plateau in the reactions, it was not surprising that boiling seemed to exert no effect on it in this experiment. In summary, we have serendipitously found that boiling promoted the inhibitory activity of BAM towards CFI.

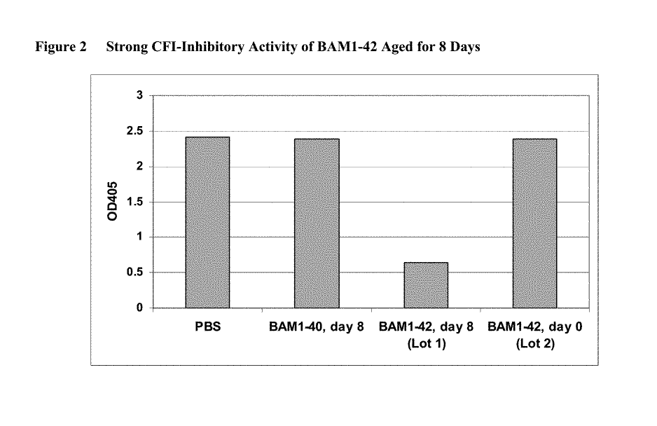

The second approach to search for the negative control of BAM yielded the expected results. The freshly made BAM1-42 (Lot 2) was shown to have no appreciable activity, whereas BAM1-42 (Lot 1) at day 8 still had strong inhibitory activity as those demonstrated at day 2 or day 3 (FIG. 2). Therefore, BAM1-42 can be considered as an inhibitor of CFI bioactivity.

### 2.6 Sonication Enhanced the Activity of BAM1-42

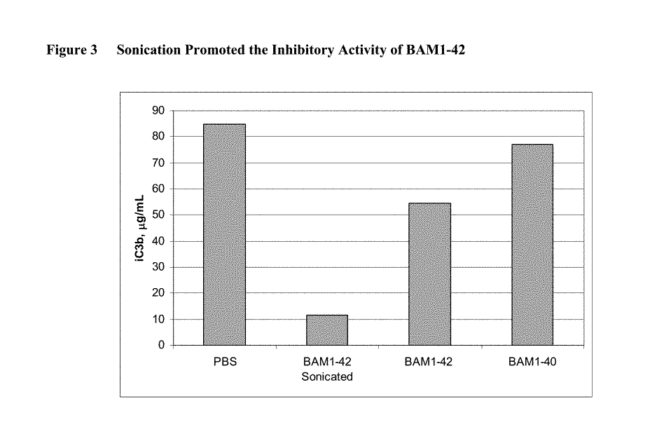

BAM1-42 (Lot 2) was not active when it was freshly made. Furthermore, when this Lot 2 BAM 1-42 was tested after ageing for 7 days, it inhibited the CFI bioactivity for only about ˜25% for CFI at 10 μg/mL, whereas the Lot 1 BAM 1-42 inhibited the CFI bioactivity almost 100% when tested at day 3 and day 8 (42). BAM 1-42 (Lot 2) was made into solution based on the main procedures for making the Lot 1 solution, except that sonication was not applied to Lot 2. To test whether sonication played any role in the enhancement of the activity, the BAM1-42 (Lot 2 aged for 9 days) solution was divided into two aliquots. One aliquot was sonicated for 15 min in Branson 1210 sonicator, while the second one was undisturbed. Both sonicated and non-sonicated BAM1-42 solutions, along with BAM1-40, were tested in the cofactor assay. The results showed unequivocally that sonication was important for BAM to form active structures that inhibit CFI bioactivity (FIG. 3).

### 2.7 CFI Dose Response in the Presence of BAM1-42

We have demonstrated that BAM 1-42 inhibits the ability of CFI to cleave C3b into iC3b. An important question to address would be how much of the CFI bioactivity is being affected by BAM 1-42 and how to quantify the apparent loss of bioactivity. To answer these questions, a CFI dose response study was conducted where CFI was tested in a range of 10 ng/mL to 30 μg/mL in the presence or absence of 200 μM BAM1-42 (Lot 2, aged for 7 days without sonication) in the cofactor assay. The results of this study showed that BAM 1-42 reduced the bioactivity of CFI about 5 fold at EC50 compared to the PBS control (EC50s for BAM 1-42: 3324.5 ng/mL; for PBS: 686 ng/mL).

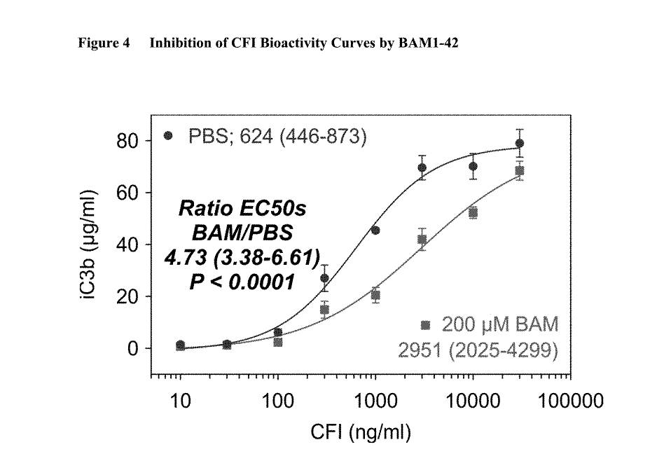

An additional analysis of the same data was conducted by the applicant using a reparametrized 4-parameter logistic equation as described in Ghosh et al, 1998. As already stated above, the concentration dependent conversion of C3b into iC3b was reduced by preincubation of CFI with 200 μM BAM1-42 (FIG. 4). The amount of CFI needed to cleave 50% of the available C3b was approximately 632 ng/ml. In contrast, after preincubation with BAM1-42, approximately 3200 ng/ml CFI equivalent were needed to achieve the same rate of cleavage. This results in a net, BAM1-42-dependent, 5-fold reduction in CFI bioactivity, which appears to be statistically significant because there is no overlap in the 95% confidence limits of the estimated EC50s (FIG. 4, numbers between parentheses).

### Conclusion of Feasibility Study

In this study, we have established that both BAM1-40 and BAM1-42, when prepared and aged properly, can inhibit the bioactivity of CFI to cleave C3b into iC3b while CFH is added as a cofactor. In addition, this set of experiments establish the basis for a quantitative cofactor assay that can effectively measure bioactivity of CFI (evaluated in this report), but also potentially the bioactivity of CFH.

The BAM-dependent reduction of CFI bioactivity can be rapidly and accurately quantified using this methodology because of the quantitative nature of the ELISA methodology employed to detect the end product of the reaction. This contrasts with the best qualitative methodologies, i.e., Western blot or SDS-PAGE techniques documented in the literature. We have also shown that the inhibitory activity of BAM1-42 was much more pronounced than that observed for BAM1-40. Various treatments, such as sonication, boiling, and ageing at 4° C., significantly influence the activity of BAM as measured in terms of blockade of CFI bioactivity. The in vitro cofactor assay conditions have been fine-tuned such that cleavage of C3b could be controlled with different efficiencies, which will provide extreme flexibility moving forward in our attempts to establish specific assay configurations for different applications.

TABLE 1
Enhanced Inhibitory Activity of BAM Aged for 3 days
Cofactor Assay Conditions

| | | | |
|---|---|---|---|
| CFI | 1 μg/ml | 10 μg/ml | 10 μg/ml |
| CFH | 10 μg/ml | 10 μg/ml | 80 μg/ml |

Results

| Inhibitors | Percent Inhibition of iC3b Generation | | |
|---|---|---|---|
| PBS | 0.0 | 0.0 | 0.0 |
| 200 μM BAM 1-40, day 3 | 23.9 | 11.3 | 10.7 |
| 200 μM BAM 1-42, day 3 | 98.8 | 95.9 | 86.3 |

Part II - Demonstrating that our ELISA assay can measure CFI bioactivity in AMD patient's in vitreous fluid, and CFI bioactivity of plasma of Alzheimer's patient before and after administration of an anti-BAM antibody

### Source of Materials

Factor I, Factor H, C3b, and iC3b were purchased from Complement Technology, Inc (Tyler, Tex.). MicroVue iC3b EIA kit (ELISA kit) was purchased from Quidel Corp (Santa Clara, Calif.). ELISA kit for Complement factor I was purchased from USCN Life Science (Wuhan, China). Beta amyloid recombinant peptide (1-42) (Ultra Pure, TFA Form) was purchased from Covance.

The following anti-amyloid antibodies and control antibodies were used in the studies.

| Clone | Isotype | Specificity | Source |
|---|---|---|---|
| 4G8 | mIgG2b | 17-24 aa | Covance |
| 6E10 | mIgG1 | 1-16 aa | Covance |
| 12F4 | mIgG1 | 42^{nd} aa | Covance |
| 11A50-B10 | mIgG1 | BAM 1-40 | Covance |
| Ab5 | mIgG2b | 1-16 aa | QED Bioscience |
| Mouse IgG1 | mIgG1 | Isotype control | Invitrogen |
| Mouse IgG2b | mIgG2b | Isotype control | Invitrogen, BD |

### 3.1. Preparation of Standards

As in 2.1 the reference standard iC3b (1.1 mg/mL) was used to prepare the standards at the nominal concentrations between 5 ng/mL to 10000 ng/mL in Assay Buffer (10 mM Tris, 60 mM NaCl, 0.1% BSA, 0.1% Tween 20, pH 7.2) as assay matrix.

### 3.2. In Vitro Plasma Cofactor Assay

Similar to the in vitro cofactor assay in Section 2.4, CFH (80 μg/mL) and C3b (80 μg/mL) were mixed with PBS-diluted plasma, which was used as a source of CFI. The mixture was incubated at 37° C. for 1 to 2 hours. As negative controls, plasma samples which had been pre-diluted 10 times with PBS and then treated at 60° C. for 2 hours were included in some assays. After the incubation, the reaction mixture was used for quantification of iC3b according to the procedures described in Section 2.7.

3.3. ELISA Detection of iC3b

During the course of the study, two ELISA methods were used. In the first half of the study, the commercial kit from Quidel was used and the kit procedures were followed. We developed in house ELISA method for detection of iC3b. Briefly, a 96-well plate was coated with monoclonal anti-human iC3b antibody (Quidel Cat No.: A209) and incubated overnight at 4° C. The plate was then washed 5 times with 300 μL of wash buffer per well using a plate washer, and each well was filled with 200 μL of blocking buffer. The plate was incubated for 1 hour at RT with shaking and washed again 5 times. One hundred microliters of Assay Buffer-diluted standards and samples were added to the appropriate wells and incubated with shaking for 1 hour at RT. The plate was then washed 5 times. One hundred microliters of HRP-conjugated anti-human C3c (AbD Serotec Cat No.: 2222-6604P) was added to each well and incubated with shaking for 1 hour at RT. The plate was then washed 5 times. One hundred microliters of Ultra-TMB ELISA Substrate (Thermo Cat No. 34028) was added and incubated for 10 to 30 min with shaking at RT and the reaction was stopped by adding 100 μL of 2M Sulfuric Acid per well. The absorbance was then measured at 450 nm. The data was processed using Gen 5™ software.

### 3.4. ELISA Detection of CFI

CFI protein concentration in human plasma was determined using a commercial ELISA kit for human CFI from USCN Life Science. Eight human plasma samples, four of which were complement grade and were derived from individual donors and prepared from blood samples immediately after blood was drawn (from Bioreclamation). Human CFI concentration in these plasma samples was measured using the ELISA kit by following the procedures provided in the kit. As a control, purified human serum CFI protein, purchased from Complement Technology (Tyler, Tex.), was also included in the test.

### 3.5. Major Computer Systems and Data Processing

Absorption data were acquired using a BioTek ELx800 microplate reader (Winooski, Vt.), which was controlled by a Dell PC workstation via Gen5™ software (BioTek). The acquired data were processed using Gen5 software (BioTek) and Microsoft Excel 2003. A standard curve for iC3b quantification was constructed by plotting the concentrations of standards in log scale (X-axis) versus their corresponding absorbance at 405 nm or 450 nm (Y-axis), and fitted with a 4-parameter logistic algorithm. The concentration of the iC3b in the samples was determined based on the standard curve. Microsoft XLfit was used to graph the curves of the concentrations of iC3b versus the concentrations of Antibody, BAM or CFI.

### 3.6 CFI Dose and Time Course Responses

As shown in Part I, an in vitro cofactor assay has been established in which CFI converts substrate C3b into iC3b in the presence of cofactor CFH. To fully characterize the bioactivity of CFI in the assay, CFI at 30, 100, 300, and 1000 ng/mL was incubated at 37° C. with 80 μg/mL CFH and 80 μg/mL of C3b for 0.5, 1, 2, 4 and 22 hours. The concentration of the end product, iC3b, was determined using the iC3b ELISA kit. Within the incubation time of 2 hours, the iC3b produced was consistently in proportion to the concentrations of CFI within the range tested. This result guided us next to develop an in vitro plasma cofactor assay.

### 3.7. Establishment of In Vitro Plasma Cofactor Assay

The bioactivity of CFI in plasma is believed to be an important biomarker for certain diseases. However, there is no assay that precisely measures the bioactivity of CFI in plasma. Since we have developed the in vitro cofactor assay for measurement of CFI or CFH bioactivity in pure protein form, we are in a better position to develop another assay for measurement of plasma CFI bioactivity by modification of the existing method. Plasma is a complex mixture of macro- and micro-molecules. It was reported that CFI is present in circulation at a concentration of approximately 39-100 μg/ml (De Paula et al., 2003), and CFH at approximately 500 μg/mL (Rodriguez de Cordoba et al., 2004). In order to measure the bioactivity of CFI in plasma, the effect of plasma source of CFH and other molecules has to be minimized. We plan to do so by diluting plasma at least 500 fold so that CFH in the diluted plasma (<1 μg/mL) becomes negligible as compared to the exogenous CFH in the reaction (80 μg/mL). In this way, CFI in the diluted plasma will still be in the range of detection when it is used as a source of CFI since the cofactor assay described above is a very sensitive assay for CFI.

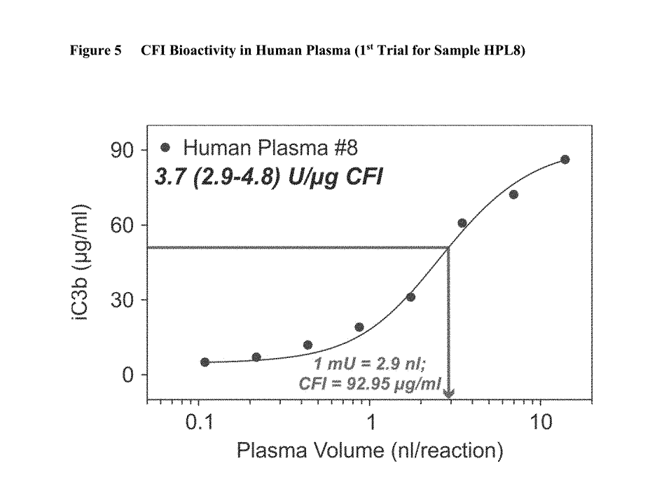

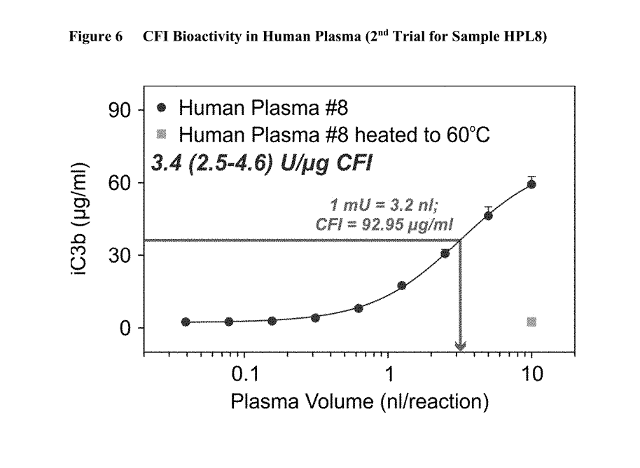

Human plasma sample from the same donor, HPL8, was tested in two independent experiments following the method described in Section 2.7. One mU of CFI bioactivity is defined in this assay as the inverse of the amount of CFI or the volume of plasma in nL needed to convert 50% C3b into iC3b in the cofactor assay. Plasma CFI bioactivity from the same donor in two experiments was 2.9 nL and 3.2 nL, respectively, according to the curves generated (FIG. 5 and FIG. 6). When defined as the Units per amount of CFI, they were 3.7 U/μg CFI and 3.4 U/μg CFI, respectively, since the CFI protein concentration in plasma was 92.95 μg/mL (see next section). Another donor's plasma sample was also tested and the CFI bioactivity was 2.3 nL or 3.6 U/μg CFI (Conc. of CFI in plasma was 123.4 μg/mL) (see next section). These results demonstrate that the method for measuring plasma CFI bioactivity is sensitive and feasible.

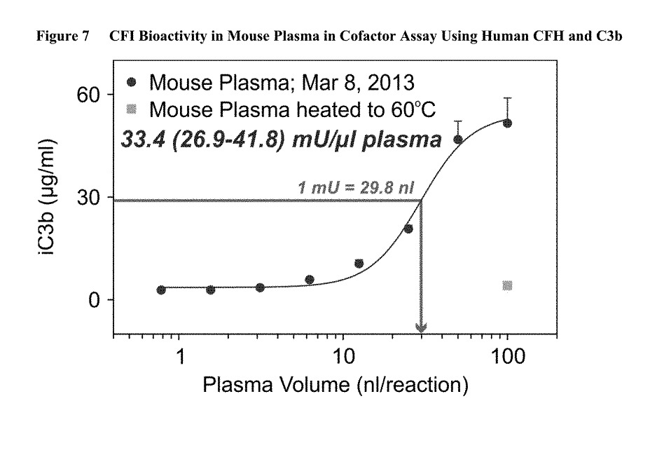

Similarly, mouse CFI bioactivity in plasma was tested following the same methodology as that used for human plasma CFI bioactivity. Since mouse CFH and C3b protein is not available, a bonafide mouse cofactor assay cannot be established. Because of the significant homology between mouse proteins and human proteins, mouse CFI might work with human CFH and C3b. As hypothesized, mouse plasma CFI indeed can convert human C3b into iC3b in the presence of human CFH albeit with 10-times less activity (FIG. 7).

### 3.8. Measurement of CFI Concentration in Human Plasma

In order to define CFI bioactivity in human plasma corrected for the amount of CFI protein present in the human plasma samples, the concentration of CFI in the plasma needs to be determined. A commercial ELISA kit for human CFI from USCN Life Science was used. CFI concentration was determined following the manufacturer's instructions in 8 human plasma samples. The concentrations of CFI in these samples fell within the reported CFI concentration range. However, purified serum CFI protein which has been successfully used in cofactor assays was unable to be detected by the kit. This raised question as to the accuracy of CFI concentration detected by the kit in the human plasma samples.

According to the manufacturer, the kit uses a monoclonal antibody raised against recombinant CFI peptide expressed in E Coli, which may explain the discrepancy above. A reliable CFI ELISA method that detects human serum CFI protein would be desirable in order to accurately quantify CFI in human plasma samples.

### 3.9. Preparation of Active BAM Solutions

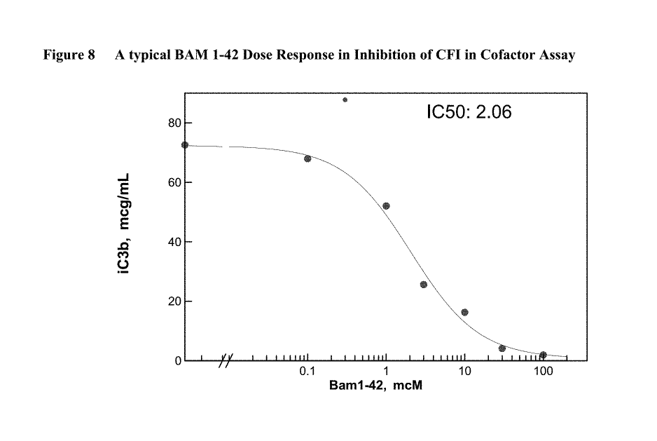

Throughout the studies, three lots of BAM 1-42 solutions have been prepared using slightly different procedures and treatments. When a BAM solution was initially made, aged for a few days at 4° C., it was then tested for activity of inhibition of CFI in cofactor assay. If not up to the activity desired, it would be aged again or treated with vigorous shaking at 37° C. for a few hours up to overnight and then the activity was tested again. Only when the IC50 reached 1˜10 μM the BAM solution was considered active and would be used for the assays. In later stage of the study, to keep the BAM activity consistent, active BAM solution was split into small aliquots and stored at −70° C. A typical BAM dose response curve is presented in (FIG. 8), with an IC50 of 2.06 μM.

### 3.10. Effect of Anti-BAM Antibodies on the Activity of BAM in the Cofactor Assay

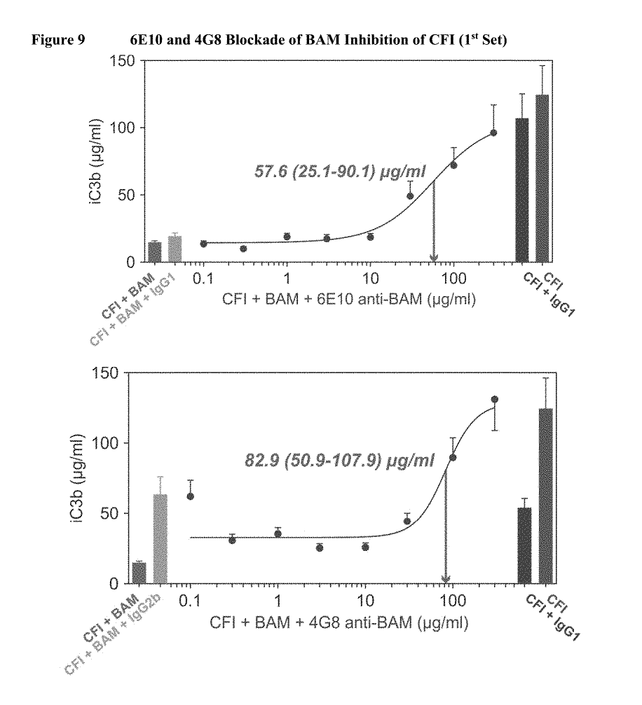

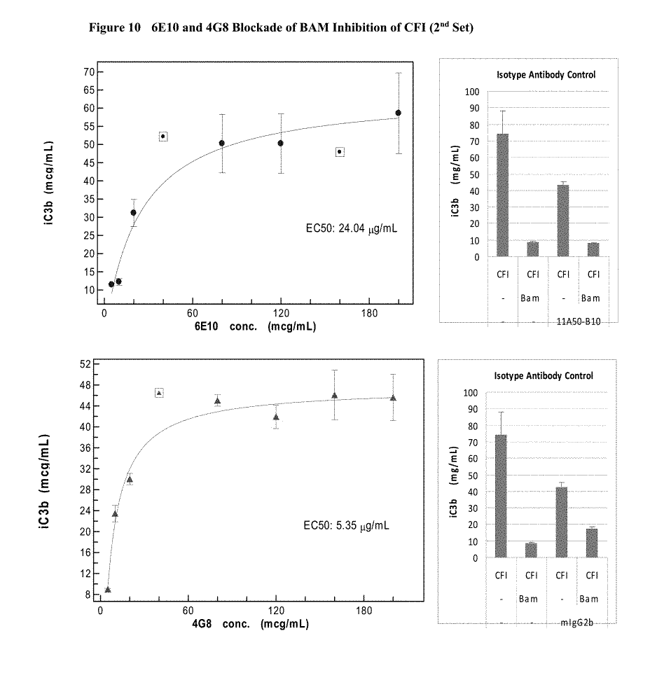

To test the effect of anti-BAM antibodies on BAM-mediated inhibition of CFI bioactivity, several commercial available mouse monoclonal antibodies targeted to different epitopes of BAM were selected. Single dose antibody at 100 μg/mL to 300 μg/mL was tested initially as described in the procedures. Among the antibodies tested, 6E10 was the strongest antibody that could reverse the activity of BAM and restore the bioactivity of CFI. Mouse isotype control antibody IgG2b could also enhance the bioactivity of CFI, causing significant background and making it difficult to differentiate the true specific anti-BAM effect. This was an issue for 4G8 which is a mouse IgG2b. Whereas this effect was less apparent with mIgG1, this isotype sometimes also gave some background. The effect of mIgG1, however, was observed much less frequently and in much smaller scale than those seen for mIgG2b. It has been reported that BAM binds to multiple molecules in the blood, including IgG (Huang et al., 1993). Because of this issue, in order to confirm the true inhibitory activity, multiple clones of anti-BAM antibodies were tested in single dose or 8-dose responses multiple times in different assay conditions. Selected results were presented in FIG. 9 and FIG. 10.

3.11. Optimization of Antibody Cofactor Assay Conditions to Minimize Non-Specific Antibody Binding with BAM

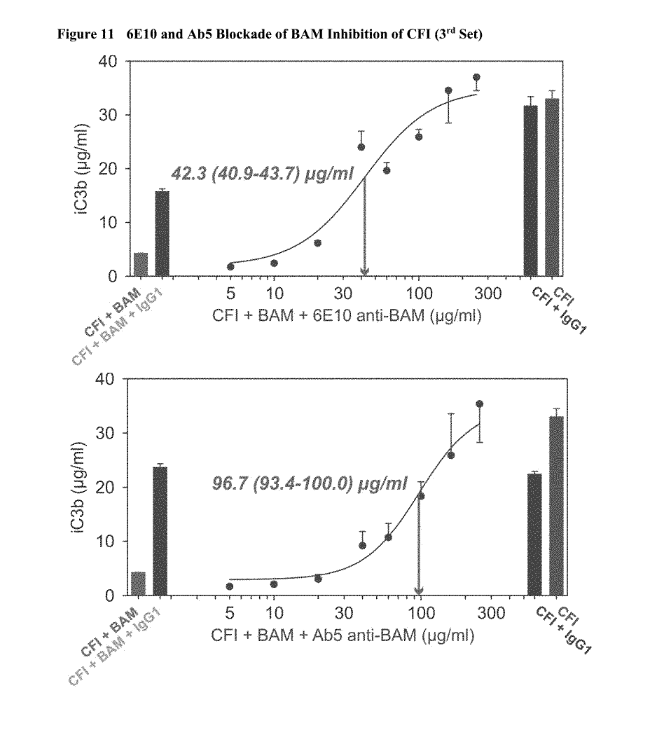

As shown in Section 3.10, mouse isotype control IgGs, especially mIgG2b, caused significant background in terms of increasing CFI bioactivity by potential interaction with BAM at the level of the hinge. To reduce such an effect, multiple agents such as Tween 20, Triton X-100, Guanidine and human serum albumin were tested in the cofactor assays to see if they will interfere with CFI bioactivity or BAM activity first. Tween 20 and Triton X-100 didn't affect CFI bioactivity, but abolished the inhibitory activity of BAM completely. Guanidine, on the other hand, significantly reduced both CFI bioactivity and the inhibitory activity of BAM. However, the effect didn't seem to be differential (FIG. 11). Taken together, none of the agents tested so far could resolve the IgG BAM binding issue. Lastly, another strategy was tested by reversing the order of addition of the reagents. Normally, BAM and anti-BAM antibody were incubated first, then CFI was added to the mixture, CFH and C3b was added last. In this experiment, BAM and CFI were incubated first, and then anti-BAM antibody was added to see if it could reverse the action of BAM on CFI bioactivity. It turned out it could, but in a much less activity that the amount of iC3b produced was less than 5 μg/mL. Nevertheless, this could serve as a starting point for further optimization.

### 3.12 Relationship Between AMD and CFI Bioactivity In Vitreous Samples

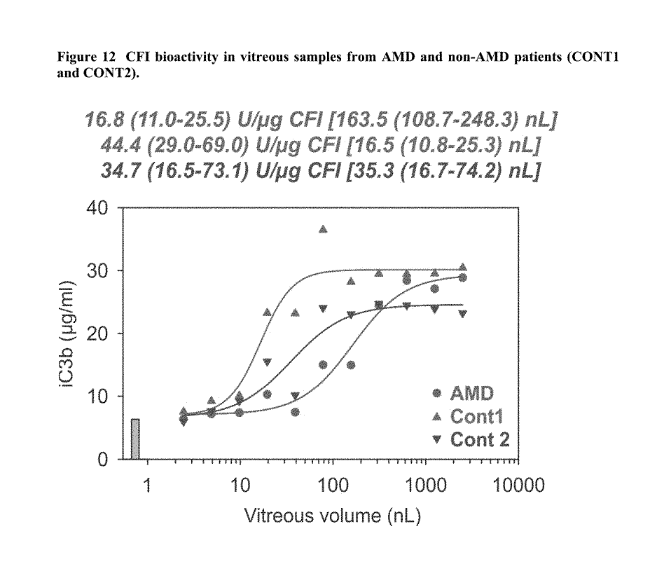

An additional experiment was conducted in which both the concentration of CFI and the bioactivity of the molecule were determined in vitreous samples from an AMD patient and from two non-AMD subjects. The concentrations of CFI were 364.65, 1363.95 and 817.36 ng/ml for the AMD and the two non-AMD patients, respectively. Evaluation of the bioactivity of the molecule using the cofactor assay revealed that the bioactivity response curve for the AMD patient was right shifted with respect to the two non-AMD patients (˜164 nL EC50 versus ˜17 and 35 nL for the AMD and the two non-AMD subjects, respectively). In terms of activity, these values translate to ˜6.1 for the AMD sample and 58.8 and 28.6 U of CFI bioactivity/ml of vitreous for non-AMD subjects. The bioactivity of CFI was corrected for the amount of the protein present in the samples, resulting in the following values: 16.8, 44.4 and 34.7 U/μg of CFI for the AMD and the two non-AMD patients (FIG. 12). These data suggest that in AMD the bioactivity of CFI is reduced when compared with that observed in non-AMD subjects, and indicate that this mechanism may be involved in the pathogenesis of the disease.

3.15. Further Confirmation that Bioactivity of CFI Differ from Sources Obtained, Further Confirming CFI Bioactivity is More Relevant Biologically Rather than the Amount

Measurements of CFI bioactivity were conducted in two samples corresponding to purified CFI and CFI that had been generated by recombinant means.

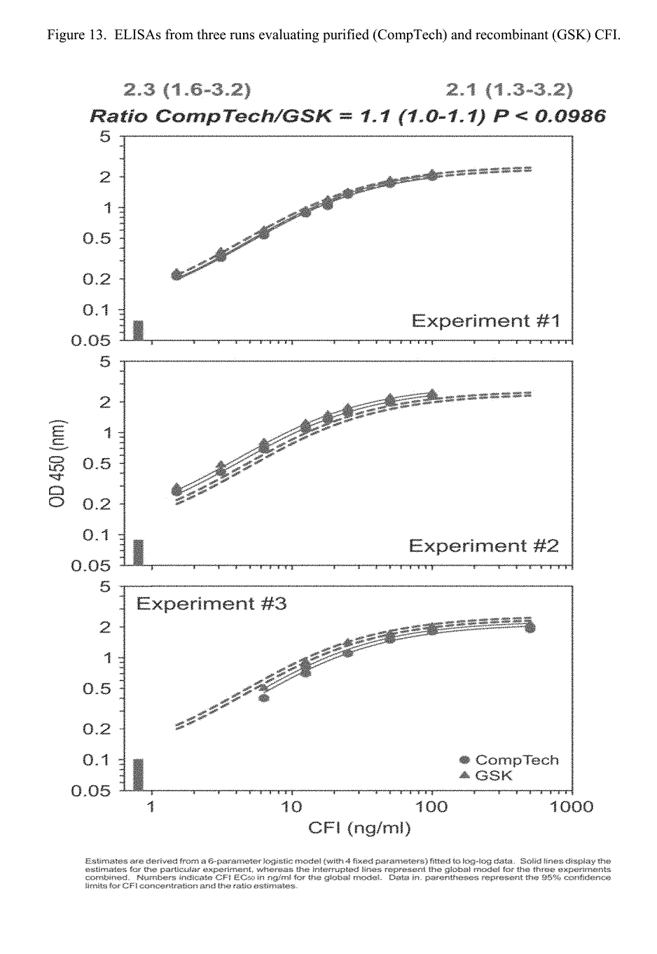

In order to confirm that both preparations had similar concentrations, we used a CFI ELISA to determine whether, from an immunoreactivity point of view, the purified and the recombinant preparations were equivalent in terms of concentration. Multiple concentrations of both purified and recombinant material were run in CFI ELISAs in duplicate and these studies were conducted on three different occasions (labeled in the figure as experiments #1 to #3. The results of these studies are shown in FIG. 13. FIG. 13 illustrates that the EC50 ratio of purified over recombinant material is approximately 1 and that this difference is not statistically significant. These data indicate that the amounts of the purified and recombinant material are nearly identical.

Further support for this notion comes from a recovery study using purified material as standard and different concentrations of recombinant material as unknown. The results from this study are summarized in the following table:

| Nominal | Observed | | | | | | | | |
|---|---|---|---|---|---|---|---|---|---|
| (ng/mL) | (ng/mL) | Mean | SEM | (n) | % CV | % Nominal | Mean | SEM | (n) |
| 60 | 83.847 | 78.52 | 7.54 | 2 | 9.6 | 139.75 | 130.86 | 12.56 | 2 |
| 60 | 73.187 | | | | | 121.98 | | | |
| 60 | 74.628 | 65.74 | 12.58 | 2 | 19.1 | 124.38 | 109.56 | 20.96 | 2 |
| 60 | 56.843 | | | | | 94.74 | | | |
| 20 | 20.436 | 20.90 | 0.65 | 2 | 3.11 | 102.18 | 104.48 | 3.25 | 2 |
| 20 | 21.355 | | | | | 106.78 | | | |
| 20 | 19.37 | 20.10 | 1.03 | 2 | 5.14 | 96.85 | 100.50 | 5.16 | 2 |
| 20 | 20.83 | | | | | 104.15 | | | |
| 5 | 4.211 | 4.27 | 0.09 | 2 | 2.07 | 84.22 | 85.47 | 1.77 | 2 |
| 5 | 4.336 | | | | | 86.72 | | | |
| 5 | 4.247 | 4.34 | 0.13 | 2 | 2.92 | 84.94 | 86.73 | 2.53 | 2 |
| 5 | 4.426 | | | | | 88.52 | | | |

Overall mean 102.93
Overall SEM 5.11
Overall (n) 12

The data in the table indicate that when using recombinant material, we recover in the assay 100% of what we added using the purified CFI as the standard, reinforcing the notion that the concentrations of protein in both preparations is equivalent.

The bioactivity measurements were conducted in triplicate and run on three occasions. Both CFI stock solutions were diluted in a ⅓ log series into 8 substock solutions starting at 60 mg/mL CFI. To maintain the CFI bioactivity in low concentration, 10 μg/mL CFH was used. Cofactor assays were set up by mixing 5 mL of 2×CFI substock solutions with 5 mL of CFH and C3b 2× mixture at a final concentration of 80 mg/mL for each reaction. The reaction mixtures were incubated at 37° C. for 1 hr, then the iC3b production in reactions was evaluated by a specific ELISA.

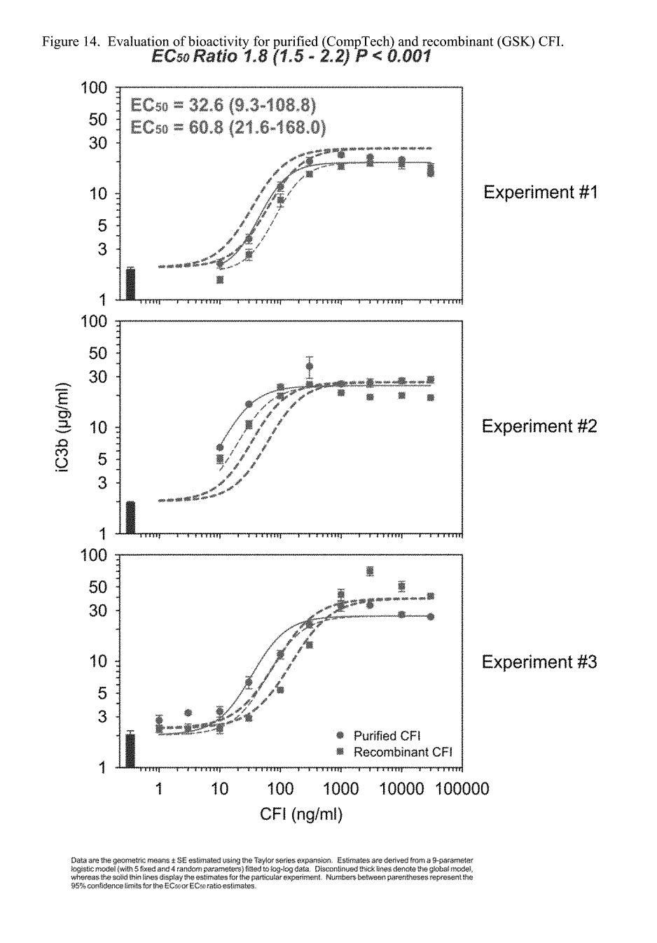

In the assay, 1 mU of CFI bioactivity is defined as the inverse of the amount of CFI that generates half of the maximal concentration of iC3b that can be produced (EC50). To obtain CFI bioactivity of a sample the following calculation is applied: 1/(EC50 of CFI in μg). This equation will result in CFI bioactivity levels expressed as U/μg of CFI. The estimated EC50s from the three experiments were 32.6 (9.3-108.8) and 60.8 (21.6-168.0) ng/ml for the purified and recombinant materials, respectively. The model fitted to the data also predicted the EC50 recombinant to purified ratio. This value was 1.8 (1.5.-2.2) and this ratio was significantly different (P<0.001) from 1 (FIG. 14).

Since the reaction volume is 10 μl, the amount of CFI present in the reaction was 100 fold lower than the estimated EC50 in ng/ml. This value provides the amount of CFI in ng that generates ½ the maximal amount of iC3b. Dividing these amounts by 1000 we convert the numbers to μg. By doing the inverse estimated EC50 of CFI in μg, we obtain the estimated bioactivity for both samples: Purified CFI bioactivity=3,067.5 (919.1-10,752.7); recombinant CFI bioactivity=1,644.7 (585.2-4,629.6) U/μg of CFI.

These data prove that one can have the same concentration of a protein, while the bioactivity of the protein can be altered. Overall, the results indicate that recombinant CFI bioactivity is approximately 2 times lower than the material purified from serum, while the concentrations of the proteins are equivalent.

3.14. Providing Evidence that an Anti-BAM Antibody can Modulate CFI Bioactivity in Alzheimer's Patients.

Finally, a set of human samples from Alzheimer disease patients treated with Compound A were evaluated for CFI bioactivity in a single assay. The assay conditions follow:
80 μg/ml CFH and C3b.
0.01-25 nL of plasma (half-log dilutions).
Incubation 2 hours.
Measurement of iC3b via ELISA.
Samples from one patient were run in a single assay.
Each assay was run in singlets.
To control inter-assay variability a common plasma sample was run in each assay.

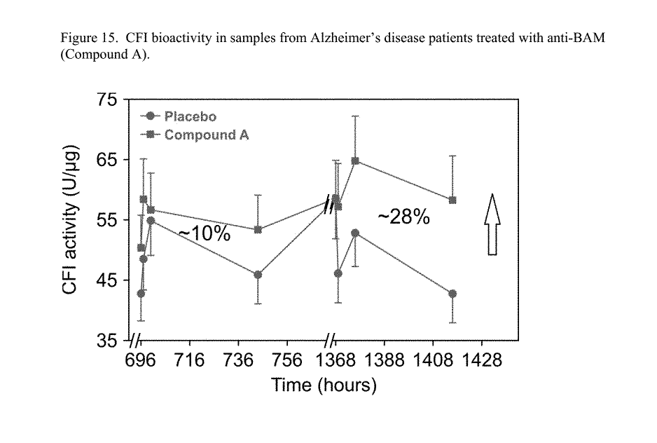

FIG. 15 shows the results of this evaluation. Compound A when administered as a single dose (6 mg/kg) at time 695 and 1367 intravenously induced an overall increase of CFI bioactivity of 10 and 28% after the second and third administration of the agent, respectively. The data suggest that anti-BAM treatment modulate CFI bioactivity in Alzheimer's disease patients.

### CONCLUSIONS

In Part I of the study, we established a method for measuring CFI bioactivity in human and mouse plasma. In Part II, we demonstrated that several anti-BAM antibodies can inhibit BAM activity by reversing CFI bioactivity back to the original bioactivity. Further, the data from vitreous samples from AMD and non-AMD patients provide support to the notion that in AMD the bioactivity of CFI is reduced and this reduction may be responsible for the activation of the alternative complement cascade that is observed in AMD eyes. Finally we have demonstrated that anti-BAM antibody will restore CFI bioactivity in plasma in beta-amyloid implicated diseases, such as Alzheimer's disease.

### LIST OF ABBREVIATIONS

BAM or Bam Beta-amyloid peptide
BAM1-40 Beta-amyloid Peptide from 1 to 40
BAM1-42 Beta-amyloid Peptide from 1 to 42
% Bias Difference between measured value and nominal value expressed as a percentage
CFH Complement factor H
CFI Complement factor I
Conc. Concentration
EC50 Half maximal effective concentration
ELISA Enzyme-linked immunosorbant assay
HRP Horse radish peroxidase
IC50 Half maximal inhibitory concentration
mcg/mL Microgram per milliliter
μg/mL Microgram per milliliter
μL Microliter
mL Milliliter
mcM Micromolar
μM Micromolar
OD405 Absorption at 405 nm
RT Room temperature
STD Standard

### REFERENCES

Bertoncini C W, Celej M S. Small molecule fluorescent probes for the detection of amyloid self-assembly in vitro and in vivo. Curr Protein Pept Sci. 2011 May; 12(3):205-20.
Bradt B M, Kolb W P, Cooper N R. Complement-dependent proinflammatory properties of the Alzheimer's disease beta-peptide. J Exp Med. 1998 Aug. 3; 188(3):431-8.
Brandstätter H.; Schulz P., Polunic I.; Kannicht C., Kohla G., Römisch J. Purification and biochemical characterization of functional complement factor H from human plasma fractions. Vox Sanguinis 2012 October; 103(3):201-12.
De Paula P F, Barbosa J E, Junior P R, Ferriani V P, Latorre M R, Nudelman V, Isaac L. Ontogeny of complement regulatory proteins—concentrations of factor h, factor I, c4b-binding protein, properdin and vitronectin in healthy children of different ages and in adults. Scand J Immunol. 2003 November; 58(5):572-7.
Ghosh K, Shen E S, Arey B J, Lopez F J. A global model to define the behavior of partial agonists (Bell-shaped dose-response inducers) in pharmacological evaluation of activity in the presence of the full agonist. J of Biopharmaceutical Statistics, 8(4), 645-665 (1998).
Jiang D, Rauda I, Han S, Chen S, Zhou F. Aggregation pathways of the amyloid β(1-42) peptide depend on its colloidal stability and ordered β-sheet stacking. Langmuir. 2012 Sep. 4; 28 (35): 12711-21.
Rodriguez de Cordoba S, Esparza-Gordillo J, Goicoechea de Jorge E, et al. The human complement factor H: functional roles, genetic variations and disease associations. Mol Immunol 2004; 41:355-367
Straub J E, Thirumalai D. Toward a molecular theory of early and late events in monomer to amyloid fibril formation. Annu Rev Phys Chem. 2011; 62:437-63.
Uversky V N. Mysterious oligomerization of the amyloidogenic proteins. FEBS J. 2010 July; 277(14):2940-53.
Wang J, Ohno-Matsui K, Yoshida T, Kojima A, Shimada N, Nakahama K, Safranova O, Iwata N, Saido T C. Mochizuki M, Morita I. Altered function of factor I caused by amyloid beta: implication for pathogenesis of age-related macular degeneration from Drusen. J Immunol. 2008 Jul. 1; 181(1):712-20.
Huang D, Martin M, Hu D, Roses Ad, Goldgaber D, Strittmatter W j. Binding Of IgG To Amyloid Beta A4 Peptide via the Heavy-Chain Hinge Region with Preservation of Antigen Binding. J Neuroimmunol. 1993, 48(2):199-203

### Additional Protein Sequences Referred in the Specification

CAA51135 light chain acceptor framework V region
amino acid sequence
(SEQ ID No: 7)
DIVMTQSPLSLPVTPGEPASISCRSSQSLLHSNGYNYLDWYLQKPGQSPQL
LIYLGSNRASGVPDRFSGSGSGTDFTLKISRVEAEDVGVYYCMQALQTPWT
FGQGTKVEIK
Humanised heavy chain V region variant H2 amino
acid sequence
(SEQ ID No: 8)
EVQLVESGGGLVQPGGSLRLSCAVSGFTFSDNGMAWVRQAPGKGLEWVSFI
SNLAYSIDYADTVTGRFTISRDNAKNSLYLQMNSLRAEDTAVYYCVSGTWF
AYWGQGTLVTVSS
Humanised light chain V region variant L1 amino
acid sequence
(SEQ ID No: 9)
DIVMTQSPLSLPVTPGEPASISCRVSQSLLHSNGYTYLHWYLQKPGQSPQL
LIYKVSNRFSGVPDRFSGSGSGTDFTLKISRVEAEDVGVYYCSQTRHVPYT
FGGGTKVEIK
Mature H2 heavy chain amino acid sequence, (Fc
mutated double mutation bold)
(SEQ ID No: 10)
EVQLVESGGGLVQPGGSLRLSCAVSGFTFSDNGMAWVRQAPGKGLEWVSF
ISNLAYSIDYADTVTGRFTISRDNAKNSLYLQMNSLRAEDTAVYYCVSGT
WFAYWGQGTLVTVSSASTKGPSVFPLAPSSKSTSGGTAALGCLVKDYFPE
PVTVSWNSGALTSGVHTFPAVLQSSGLYSLSSVVTVPSSSLGTQTYICNV
NHKPSNTKVDKKVEPKSCDKTHTCPPCPAPELAGAPSVFLFPPKPKDTLM
ISRTPEVTCVWDVSHEDPEVKFNWYVDGVEVHNAKTKPREEQYNSTYRWS
VLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSF
FLYSKLTVDKSRWQQGNVFSCSVMHEALHNHYTQKSLSLSPGK
Mature Light chain amino acid sequence
(SEQ ID No: 11)
DIVMTQSPLSLPVTPGEPASISCRVSQSLLHSNGYTYLHWYLQKPGQSPQ
LLIYKVSNRFSGVPDRFSGSGSGTDFTLKISRVEAEDVGVYYCSQTRHVP
YTFGGGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

1. A method of quantitatively measuring the CFI bioactivity of a human body fluid or the bioactivity of complement factor I (CFI) in a human body fluid comprising the steps of measuring the ability of body fluid or CFI to convert C3b to iC3b.

2. A method of quantitatively measuring the CFI bioactivity of a human body fluid or the bioactivity of CFI in a human body fluid comprising the steps of
(a) diluting the human body fluid with a diluent;
(b) adding complement factor H (CFH) and C3b to the diluted human fluid;
(c) incubating the resultant solution of step (b); and
(d) measuring the amount of iC3b generated.

3. The method of claim 2 in which the human body fluid is plasma and the diluent is PBS.

4. The method of claim 2 wherein the human body fluid is human plasma and
(a) the plasma is diluted about 200-250 fold in PBS and then further diluted in 2× series in PBS;
(b) 6 μ/L of CFH and C3b is added to the plasma to reach final concentration of 80 μg/mL each; and
(c) incubation of step (c) in claim 2 is carried out at 37° C. for 2 hr; and
(d) amount of iC3b is measured using ELISA.

5. The method of claim 4 in which the CFI bioactivity of human plasma is measured in mU per volume, in which mU is defined as the inverse of the volume of plasma in nL (EC50) needed to convert 50% C3b into iC3b.

6. The method of claim 4 in which the bioactivity of CFI in plasma is derived in terms of Units per amount of μg of CFI using the following equation of 1/(EC50 (in nL)×1000, wherein EC50 is the volume of plasma in nL needed to convert 50% C3b into iC3b.
